(19) United States
(12) Patent Application Publication
(10) Pub. No.: US 2017/0340237 A1
(43) Pub. Date: Nov. 30, 2017

(54) ELECTRODE TAPE

(71) Applicant: Timpel S.A., Pinheiros, São Paulo-SP (BR)

(72) Inventors: Rafael Holzhacker, Pinheiros (BR); Igor Nowaski Candiani, Jardim Vazani (BR)

(73) Assignee: Timpel S.A.

(21) Appl. No.: 15/534,962

(22) PCT Filed: Dec. 11, 2015
(86) PCT No.: PCT/BR2015/050246

§ 371 Date: Jun. 9, 2017

(30) Foreign Application Priority Data
Dec. 12, 2014 (BR) BR102014031270-6

(51) Int. Cl. A61B 5/053; A61B 5/0408; A61B 5/00

(52) U.S. Cl. CPC A61B 5/0536; A61B 5/6831; A61B 5/0408; A61B 5/6823; A61B 2560/04

(57) ABSTRACT

The present invention is related to electrode belts and, more particularly, to electrode belts used to obtain signals from electrical impedance tomography. The electrode belt for acquiring signals of electrical impedance tomography comprises at least one module, in which each module comprises at least two electrodes, wherein the center of each electrode is placed at a predetermined distance in relation to the center of at least one other adjacent electrode.

### CROSS-REFERENCE TO RELATED APPLICATIONS

This application is a national phase entry under 35 U.S.C. §371 of International Patent Application PCT/BR2015/050246, filed Dec. 11, 2015, designating the United States of America and published as International Patent Publication WO 2016/090450 A1 on Jun. 16, 2016, which claims the benefit under Article 8 of the Patent Cooperation Treaty to Brazilian Patent Application Serial No. BR 102014031270-6, filed Dec. 12, 2014.

### TECHNICAL FIELD

The present disclosure relates generally to electrode belts and, particularly, to electrode belts used to obtain electrical impedance tomography (EIT) signals.

### BACKGROUND

Within the techniques of image reconstruction known in the state of the art, four stand out: magnetic resonance imaging (MRI), CT scan, medical ultrasound, as well as the electrical impedance tomography (EIT).

Although some image reconstruction techniques provide a better spatial resolution of images than those generated by electrical impedance tomography (EIT), the EIT has many advantages, such as: lower cost, high temporal resolution of images, not exposing the patient to radiation risks, and smaller-sized equipment.

As a result, besides encouraging parsimony and safety, the techniques of electrical impedance tomography allow the equipment to easily be taken to the patient, so that the patient does not have to be moved from the bed to be examined.

The electrical impedance tomography (EIT) is an image acquisition technique, usually aimed at the thoracic region of the patient, based on the application of alternating electrical signals with frequencies from 10 kHz to 2.5 MHz, using electrodes attached to the patient's body surface.

“Patients” are any human being or animal. From this perspective, the above-mentioned techniques aimed at the thoracic region of the patient are generally related to the thorax, the area that extends from the base of the neck to the diaphragm of a human being or animal.

On the whole, the equipment used for this purpose comprises a plurality of electrodes placed in contact the skin. They are connected, by means of electrical conductors, to a processing unit that makes the mentioned alternating signal.

Preferably, the signals are injected through a first pair of electrodes selected from the plurality of electrodes, they run through the patient and, then, are acquired up by the other electrodes so the induced tension can be measured. Then, the previous procedure is repeated using a second pair of electrodes selected from the plurality of electrodes for the injection of the signal; following this sequence until all electrodes of the equipment have been selected, thus completing one exploring cycle.

The induced tensions that were acquired by the electrodes undergo a specific software treatment, allowing image generation, which usually represents the ventilation and perfusion phenomena in the organism observed.

The electrodes are usually held by a belt, which is placed around the body of the patient, preferably in the area of the thorax. In this regard, it is relevant to mention the document PI 0704408-9 that describes modular belts that have a plurality of electrodes, to be applied around a part of the body of a human or animal patient.

One of the known problems of the state of the art is related to the difficulty in setting up the electrodes on the patient, because it is very laborious and the patient has to be lifted from the bed.

Another problem known in the state of the art is related to pressure ulcers (wounds) on the patient's skin, caused by sensors and electrodes that are thick, of irregular surface, of variable thickness, that apply excessive and continuous pressure on the patient's skin, especially in situations where the patients are lying on or in direct contact with the thick parts of the belt, or even the cords that connect the belt to the equipment.

As seen, despite apparently being functional up to the present moment, the belts in the state of the art show some inconveniences and limitations concerning the electrode's attachment and placing.

Therefore, the present disclosure's objective is to provide an electrode belt for electrical impedance tomography that solves the problems known to the state of the art, in order to further improvements in the fixation and placing of the electrodes on the patient.

### BRIEF SUMMARY

In order to avoid the inconveniences of the state of the art and meet the goals above mentioned, among others, the disclosure is an electrode belt for acquiring signals of electrical impedance tomography that comprises at least one module, in which each module comprises at least two electrodes. The center of each electrode is placed at a predetermined distance (De) in relation to the center of at least one other adjacent electrode.

According to the additional or alternative embodiments of the disclosure, the following characteristics, alone or in possible technical combinations, may also be present:
each module is of a predetermined size within a first, second, third, fourth, fifth or sixth size;
the predetermined distance De for a first size module is from 19.3 mm to 21.3 mm;
the predetermined distance De for a second size module is from 23.1 mm to 25.1 mm;
the predetermined distance De for a third size module is from 25.9 mm to 27.9 mm;
the predetermined distance De for a fourth size module is from 29.0 mm to 31.0 mm;
the predetermined distance De for a fifth size module is from 32.4 mm to 34.4 mm;
the predetermined distance De for a sixth size module is from 36.2 mm to 38.2 mm;
the distance between the electrodes of each module is smaller than 10% of the perimeter of the patient's thorax;
two modules attachable to the patient's body, wherein the distance between the electrode closer to one of the edges of the module and the electrode closer to one of the edges of the adjacent module is smaller than 10% of the perimeter of the patient's thorax;
the distance between the electrodes on the patient's sternum area is smaller than 10% of the perimeter of the thorax.
the contact resistance of each electrode is lower than 100 Ohms;
each electrode's area is larger than 300 mm²;
each electrode is coated with conductive silicone;
the area between electrodes is coated with non-conductive silicone;
the non-conductive silicone coating is even along the belt, wherein the level variation of the conductive and non-conductive silicone surfaces is smaller than 2 mm;
each electrode is connected to a signal conductor cable;
it comprises a cable outlet placed in an area of the module located between half of the total length and one end of the belt;
each module contains 16 electrodes;
it comprises a cable outlet located in an area between the 5th and 8th electrode; and
it comprises a cable outlet located in an area between the 9th and 12th electrode;
the thickness of the belt is smaller than 6 mm.

### BRIEF DESCRIPTION OF THE DRAWINGS

The objectives, advantages, technical and functional improvements of the disclosure will be better understood with the analysis of the description of preferred embodiments, made following with regard to the figures attached, which illustrate non-restrictive preferred embodiments, in which:

FIG. 1 shows a front elevational view of an electrode belt as the first embodiment of the disclosure;

FIG. 2 shows a front elevational view with a longitudinal sectional side view of the electrode of FIG. 1;

FIG. 3 shows a front elevational view of an electrode belt as the second embodiment of the disclosure; and

FIG. 4 shows a perspective view of a module's part of the electrode belt as the third embodiment of the disclosure.

### DETAILED DESCRIPTION

The disclosure is now described regarding its preferred embodiments, referring to the attached figures. In the figures and description ahead, similar parts are marked with equal reference numbers. The figures are not, necessarily, in scale. That is, certain characteristics of the disclosure may be shown with exaggeration of scale or schematically, as well as some details of conventional elements may not be shown in order to illustrate this description in a more clear and concise way. The present disclosure is sensitive to embodiments in different ways. Specific embodiments are described in details and shown in the figures, with the understanding that the description must be considered an example of the principles here revealed, and the purpose is not to limit it only to what is illustrated and described in this descriptive report. We must acknowledge that the different teachings of the embodiments discussed next may be separately employed or in any appropriate combination to provide the same technical effects.

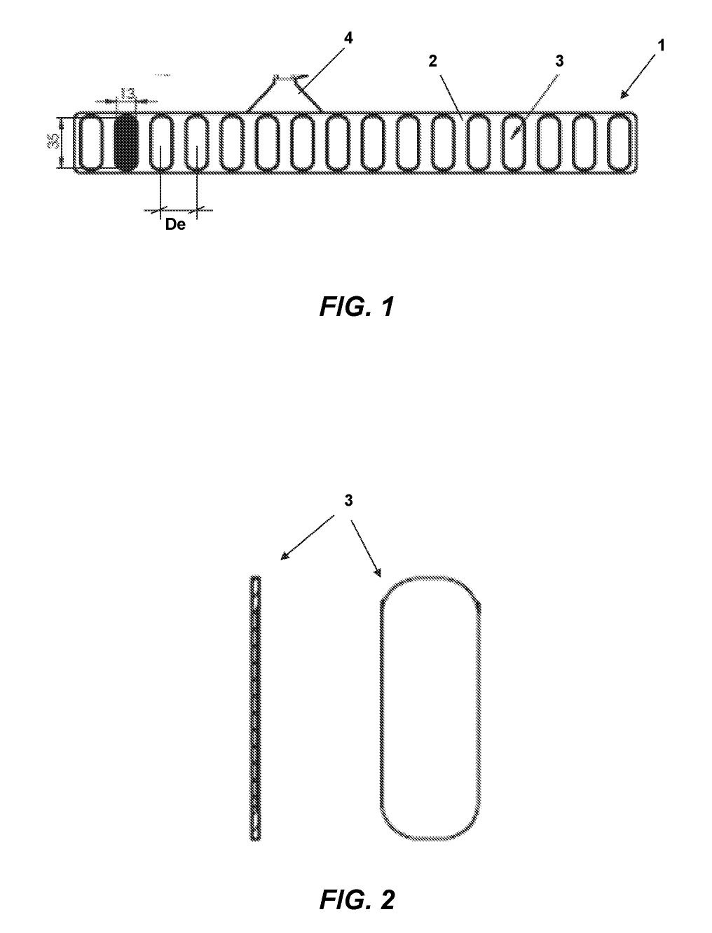

The present disclosure comprises an electrode belt, which, as it may be observed on FIG. 1, comprises a module 1 with 16 electrodes 3 placed equidistantly, that is, a distance from one electrode 3 to another adjacent is the same to any other electrode 3.

The electrode belt is particularly designed to acquire signals of electrical impedance tomography (EIT), therefore, the electrode belt is usually positioned in the area of the patient's thorax.

As it is known in the art, the size and proportion of the belt should vary according to the size of the patient that will use it. Therefore, the present disclosure expects the use of at least five predetermined sizes, here named as: first size, second size, third size, fourth size, fifth size and sixth size. In a preferred embodiment, these sizes may have common names used in the market, such as: XS, S, M, L and XL.

Thereby, the provisions of the present disclosure allow the use of the belt on a large selection of thorax's sizes, since the use of the appropriate sized belt, according to the patient's thorax, and the specific placement of the electrodes, ensure that the distance between the electrodes 3 will be a maximum of 10% of the thorax's size.

For example, the sizes of the present disclosure's belt may be determined as: the predetermined distance De for a first size module is from 19.3 mm to 21.3 mm; the predetermined distance De for a second size module is from 23.1 mm to 25.1 mm; the predetermined distance De for a third size module is from 25.9 mm to 27.9 mm; the predetermined distance De for a fourth size module is from 29.0 mm to 31.0 mm; the predetermined distance De for a fifth size module is from 32.4 mm to 34.4 mm; and the predetermined distance De for a sixth size module is from 36.2 mm to 38.2 mm.

In this particular process, the predetermined distance De between the center of the 1st electrode and the center of the 16th electrode on a first size module is from 300 mm to 310 mm; the predetermined distance the predetermined distance between the center of the 1st electrode and the center of the 16th electrode on a second size module is from 357 mm to 367 mm; the predetermined distance De between the center of the 1st electrode and the center of the 16th electrode on a third size module is from 399 mm to 409 mm; the predetermined distance De between the center of the 1st electrode and the center of the 16th electrode on a fourth size module is from 445 mm to 455 mm; the predetermined distance De between the center of the 1st electrode and the center of the 16th electrode on a fifth size module is from 496 mm to 506 mm; the predetermined distance De between the center of the 1st electrode and the center of the 16th electrode on a sixth size module is from 553 mm to 563 mm;

In an embodiment of the disclosure, module 1 has a cable outlet 4 placed in an area of module 1 comprised between half of the total length and one end of the belt. This setting has the objective to allow the cable outlet 4 to stay between the patient's sternum and the axilla.

In other words, the placement of the cable outlet 4 is well set so that, when the belt is fastened on the patient, the cables 5 are closer to the sternum than to the spine of the patient, to facilitate access to the cables and avoid pressure ulcers caused by the patient's pressure when lying on the cables.

FIG. 1 shows an example of placement of the outlet 4 for signal transmission cable 5, which covers an area that comprises from the 5th to the 8th electrode.

FIG. 2 shows an electrode in accordance with an embodiment of the disclosure, wherein the area of the electrode 3 is larger than 300 mm², more specifically about 450 mm², represented by an element of 35 mm of length and 13 mm of width.

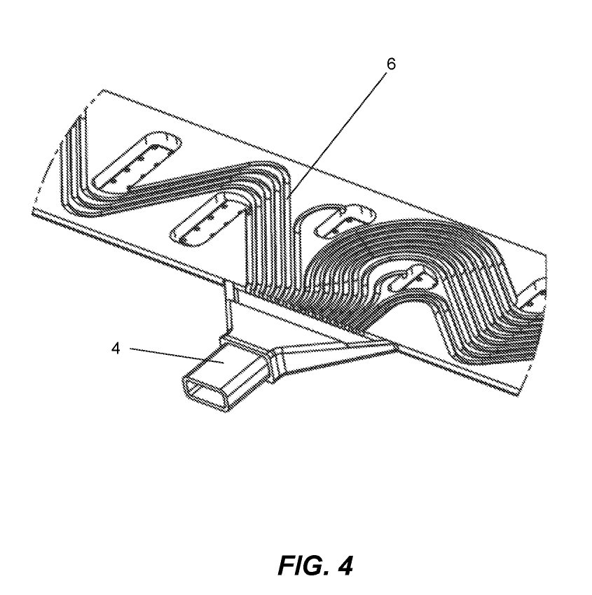

FIG. 4 illustrates another process of the disclosure, in which the non-conductive silicone has grooves 6 that work as ducts for the passage of cables 5 from electrodes 3 up to the cable outlet 4, so that the cables 5 are inside module 1, that is, the cables 5 are not in contact with the patient's skin when the belt is fastened.

In another embodiment of the disclosure, the belt consists of two modules 1, being the first module set with the cable outlet 4 placed between its half and left end, and the second module set with the cable outlet 4 placed between its half and right end, so that, when fastening the belt on the patient, the cable outlets 4 from both modules are placed on the thorax's front area. Particularly, modules 1 are placed contiguously along its longitudinal edges, around the patient's body, so that a module 1 does not overlap another and allowing all electrodes to be in contact with the patient, receiving electrical signals with no barriers. This setting of two modules 1 allows the belt to be fastened on a patient without lifting him/her from the bed.

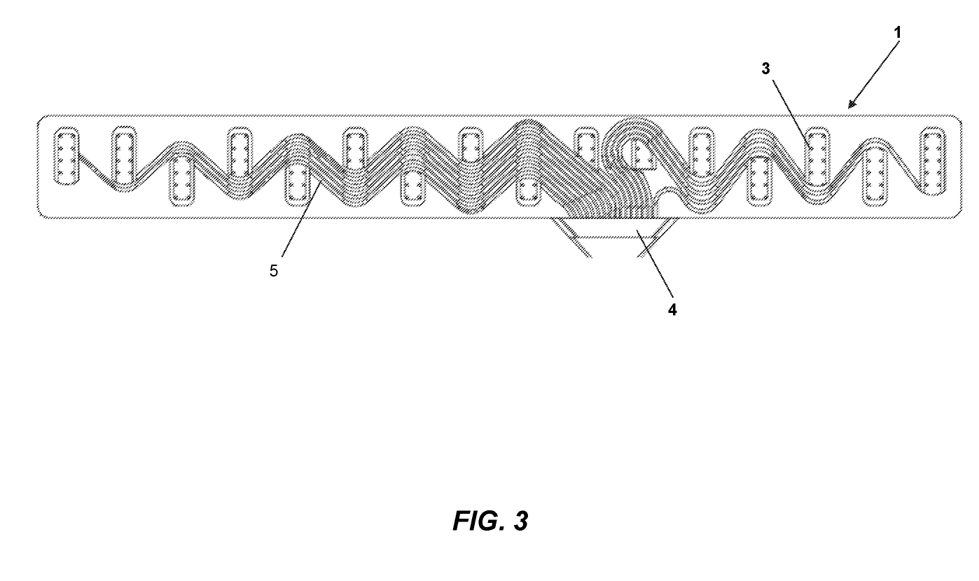

FIG. 3 illustrates another embodiment of the disclosure, in which a way of connecting the electrodes 3 to the outlet 4 of the signal transmission cable 5 is shown. The outlet 4 of the cable 5 is placed on the area that comprises electrodes 9 to 12, being the module 1 set to be used on the left side of a patient, in a way that the outlet 4 and cables 5 are placed on the front area of the patient's thorax.

Particularly, the modules 1 are coated with silicone, wherein a conductive silicone is used on the area of the electrodes 3, so that the contact resistance is lower than 100 Ohms. In other areas, mainly on an area 2 between electrodes 3, the module is filled with non-conductive silicone.

Auspiciously, the non-conductive silicone is placed in a way that the electrode belt's surface that will be in contact with the patient is flat and even, that is, with no variation between the level of the surfaces coated with the conductive silicone and the surfaces of the areas 2, covered with the non-conductive silicone.

In view of what was described above, it is visible that, auspiciously, the placement of the electrodes 3 in module 1 is done in a way that the distance 2 between two electrodes 3 is always smaller than 10% of the perimeter of the patient's thorax on the sternum area. This allows a uniform positioning and fixing of the electrodes on the patients, without leaving unwanted spaces between the electrodes, solving one of the existing problems with the state of the art.

Despite the disclosure being described regarding its preferred embodiments, it is understood that variations may occur in relation to what was described above without moving away from the scope of the disclosure. Consequently, the scope of protection is not limited to the embodiments described, but it is only limited by the following claims, which must be interpreted covering all its equivalents.

1. An electrode belt for acquiring signals of electrical impedance tomography, the electrode belt comprising at least one module comprising at least two electrodes, wherein the center of each electrode of the at least two electrodes is placed within the electrode belt at a predetermined distance (De) in relation to a center of at least one other adjacent electrode, wherein the predetermined distance (De) between each adjacent electrode across the at least one module is smaller than 10% of a perimeter of a patient's thorax for a corresponding size of the at least one module.

2. The electrode belt of claim 1, wherein the at least one module is of a predetermined size having one of a first size, a second size, a third size, a fourth size, or a fifth size.

3. The electrode belt of claim 2, wherein the predetermined distance (De) between adjacent electrodes are selected from the group consisting of:
19.3 mm to 21.3 mm, for the first size module;
23.1 mm to 25.1 mm, for the second size module;
25.9 mm to 27.9 mm, for the third size module;
29.0 mm to 31.0 mm, for the fourth size module;
32.4 mm to 34.4 mm, for the fifth size module; and
36.2 mm to 38.2 mm, for the sixth size module.

4. (canceled)

5. The electrode belt of claim 1, further comprising at least two modules connected such that when fixed on the patient's body the distance between the adjacent electrodes proximate to connecting ends of the adjacent modules is smaller than 10% of the perimeter of the patient's thorax.

6. The electrode belt of claim 5, wherein the at least two modules are placed contiguously along the longitudinal ends, such that each module does not overlap the other module.

7. The electrode belt of claim 5, wherein a resistance of contact of each electrode is lower than 100 Ohms.

8. The electrode belt of claim 7, wherein a surface area of each electrode is larger than 300 mm².

9. The electrode belt of claim 8, wherein each electrode is coated with conductive silicone.

10. The electrode belt of claim 9, wherein a region of the at least one module between the electrodes is filled with non-conductive silicone.

11. The electrode belt of claim 10, wherein the non-conductive silicone filling is uniform throughout the electrode belt such that unevenness of such that in areas filled with conductive and non-conductive silicone is smaller than 2 mm.

12. The electrode belt of claim 6, wherein each electrode is connected to a signal conductive cable in which each module includes a cable outlet placed on an area of the module between a midpoint of the total length and one end of the electrode belt.

13. The electrode belt of claim 12, wherein each module has 16 electrodes.

14. The electrode belt of claim 13, wherein the cable outlet is placed on an area between the 5th and 8th electrodes.

15. The electrode belt of claim 13, wherein the cable outlet is placed on an area between the 9th and 12th electrodes.

16. The electrode belt of claim 11, wherein a thickness of the electrode belt is smaller than 6 mm.

17. The electrode belt of claim 13, wherein the predetermined distance between the center of the 1st electrode and the center of the 16th electrode to a first size module is from 300 mm to 310 mm; the predetermined distance between the center of the 1st electrode and the center of the 16th electrode to a second size module is from 357 mm to 367 mm; the predetermined distance between the center of the 1st electrode and the center of the 16th electrode to a third size module is from 399 mm to 409 mm; the predetermined distance between the center of the 1st electrode and the center of the 16th electrode to a fourth size module is from 445 mm to 455 mm; the predetermined distance between the center of the 1st electrode and the center of the 16th electrode to a fifth size module is from 496 mm to 506 mm; the predetermined distance between the center of the 1st electrode and the center of the 16th electrode to a sixth size module is from 553 mm to 563 mm.

18. The electrode belt of claim 6, wherein each electrode is connected to a signal conductive cable in which each module includes a cable outlet placed on an area of the module that is offset from a midpoint of the total length of the electrode belt.

19. The electrode belt of claim 1, wherein the at least one module includes a first module including a first cable outlet and a second module including a second cable outlet, the first module and the second module connected such that, when fastening the electrode belt on the patient, the cable outlets from both modules are placed on a front area of the patient's thorax.

20. The electrode belt of claim 1, wherein the at least one module includes a first module including a first cable outlet and a second module including a second cable outlet, the first module and the second module connected such that, when fastening the electrode belt on the patient, the first cable outlet is offset on a left hand side of the first module and the second cable outlet is offset on a right hand side of the second module.

21. The electrode belt of claim 13, wherein the electrodes are spaced equidistantly across the at least one module.
